(19) United States
(12) Patent Application Publication
(10) Pub. No.: US 2013/0296743 A1
(43) Pub. Date: Nov. 7, 2013

(54) Ultrasound for Therapy Control or Monitoring

(71) Applicants: Chi-Yin Lee, Sammamish, WA (US); Liexiang Fan, Sammamish, WA (US); Jerry Hopple, Seattle, WA (US); Stephen J. Hsu, Issaquah, WA (US); Xiaozheng Zeng, Sammamish, WA (US); Caroline Maleke, Bellevue, WA (US); Christophe Duong, San Francisco, CA (US); Kevin Michael Sekins, Yarrow Point, WA (US); John Kook, Seattle, WA (US); Stephen R. Barnes, Bellevue, WA (US)

(72) Inventors: Chi-Yin Lee, Sammamish, WA (US); Liexiang Fan, Sammamish, WA (US); Jerry Hopple, Seattle, WA (US); Stephen J. Hsu, Issaquah, WA (US); Xiaozheng Zeng, Sammamish, WA (US); Caroline Maleke, Bellevue, WA (US); Christophe Duong, San Francisco, CA (US); Kevin Michael Sekins, Yarrow Point, WA (US); John Kook, Seattle, WA (US); Stephen R. Barnes, Bellevue, WA (US)

(73) Assignee: Siemens Medical Solutions USA, Inc., Malvern, PA (US)

(21) Appl. No.: 13/462,715

(22) Filed: May 2, 2012

(51) Int. Cl. A61N 7/00

(52) U.S. Cl. 601/3

(57) ABSTRACT

Therapy control and/or monitoring is performed with an ultrasound scanner. The ultrasound scanner detects temperature to monitor therapy, and perform HIFU beam location refocusing of the therapy system based on the temperature. The monitoring is synchronized with the therapy using a trigger output of the ultrasound scanner. The trigger output responds to a scan sequence of the ultrasound scanner. To meet a given therapy plan, the scan sequence is customized, resulting in the customized trigger sequence. Three dimensional or multi-planar reconstruction rendering is used to represent temperature for monitoring feedback. The temperature at locations not being treated may be monitored. If the temperature has an undesired characteristic (e.g., too high), then the therapy is controlled by ceasing, at least temporarily.

### BACKGROUND

The present embodiments relate to heat therapy. For example, high intensity focused ultrasound (HIFU) generates heat within a patient. In HIFU therapy, a HIFU therapy device ablates by heat, and an imaging system monitors the progress of the ablation. The imaging system displays an image, allowing the user to indicate the desired target region for therapy. In order to dose the correct regions of interest, the imaging system's coordinates are registered with the HIFU therapy device's coordinates.

In an integrated system, the therapy transducer and imaging transducer have a set or fixed relative position. The same transducer may be used for both imaging and therapy. In other arrangements, an ultrasound imaging system images and a separate therapy system applies therapy. However, there is no feedback from the imaging system to the therapy system, or a specialized communications is established for integrated operation despite separation. Specialized communications may require expensive software changes or changes in hardware.

### BRIEF SUMMARY

By way of introduction, the preferred embodiments described below include methods, computer readable media, instructions, and systems for therapy control and/or monitoring with an ultrasound scanner. Various features are used for control or monitoring of therapy by a therapy device using a separate ultrasound scanner. (1) The ultrasound scanner detects temperature to monitor therapy and perform HIFU beam location refocusing of the therapy system based on the temperature. (2) The monitoring is synchronized with the therapy using a trigger output of the ultrasound scanner. The trigger output responds to a scan sequence of the ultrasound scanner. To meet a given therapy plan, the scan sequence is customized, resulting in the customized trigger sequence. Since the ultrasound scanner is not to scan during therapy, the transmitters may be turned off even though the scan sequence otherwise configures the ultrasound scanner to scan. (3) Three dimensional or multi-planar reconstruction rendering is used to represent temperature for monitoring feedback. The user may control the rendering to assist in monitoring the volume being treated in real time with the treatment. (4) The temperature at locations being treated and/or locations not being treated may be monitored. If the temperature has an undesired characteristic (e.g., too high) outside the treated region, then the therapy is controlled by ceasing, at least temporarily. Any of theses features are used alone or in combination.

In a first aspect, a system is provided for therapy control with an ultrasound scanner. Transmitters of the ultrasound scanner are operable to scan with beams of ultrasound in a scan sequence. A processor of the ultrasound scanner is configured to create the scan sequence for the ultrasound scanner as a function of a therapy plan and to turn off the transmitters for at least a portion of the scan sequence. A trigger output of the ultrasound scanner is configured to output triggers to a therapy device. The output triggers are responsive to the scan sequence.

In a second aspect, a method is provided for therapy control with an ultrasound scanner. A treatment location is identified. A focus of a heat therapy is detected with acoustic thermometry. The focus is adjusted to be at the treatment location based on the detection with the acoustic thermometry.

In a third aspect, a non-transitory computer readable storage medium has stored therein data representing instructions executable by a programmed processor for therapy monitoring with an ultrasound scanner. The storage medium includes instructions for acquiring temperatures of tissue in locations distributed in three-dimensions, interleaving therapeutic transmissions with the acquiring of the temperatures, receiving user input of rendering manipulation, and generating, in real time with the therapeutic transmissions, a three-dimensional or a multi-planar reconstruction rendering to a two-dimensional image of the temperatures, the rendering being a function of the rendering manipulation.

In a fourth aspect, a non-transitory computer readable storage medium has stored therein data representing instructions executable by a programmed processor for therapy control with an ultrasound scanner. The storage medium includes instructions for transmitting therapeutic high intensity focused ultrasound to a treatment region within a patient region, acquiring temperatures of tissue in locations distributed in the patient region, at least some of the locations outside of the treatment region, and ceasing the transmitting of the therapeutic high intensity focused ultrasound based on at least one temperature for at least one of the locations outside the treatment region.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for therapy control and monitoring with an ultrasound imaging system;

FIG. 2 is a block diagram of one embodiment of a medical diagnostic ultrasound imaging system for controlling and monitoring a therapy device;

FIG. 3 is a block diagram of one embodiment of an arrangement of software modules for an ultrasound scanner;

FIG. 4 illustrates an example trigger sequence for controlling operation of a therapy device;

FIG. 5 is an example user interface for creating a scan sequence;

FIG. 6 shows a representation of example images of temperature associated with a focal region;

FIG. 7 is a flow chart diagram of one embodiment of a method for therapy control with an ultrasound imaging system;

FIG. 8 is a flow chart diagram of one embodiment of a method for therapy monitoring with an ultrasound imaging system; and

FIG. 9 is a flow chart diagram of another embodiment of a method for therapy control with an ultrasound imaging system.

### DETAILED DESCRIPTION OF THE DRAWINGS AND PRESENTLY PREFERRED EMBODIMENTS

During the therapy planning and treatment process, a feedback system may provide the user real-time information on how well the therapy is performed. A three-dimensional (3D) imaging ultrasound machine, such as the Siemens SC2000, performs therapy control and monitoring in conjunction with a therapy module, such as a separate HIFU therapy system. Using scan sequence creation, the ultrasound machine synchronizes the timing between therapy (e.g., HIFU) and imaging or monitoring during the course of the therapy. The ultrasound machine provides feedback to the therapy module on using acoustic thermometry, such as the location, shape, and/or size of the focus. Thermometry may be used to control delivered power. HIFU focus auto-adjustment operates based on the feedback. The ultrasound machine provides a safety mechanism to monitor the temperature outside the treated region as well as inside the treatment region. HIFU beam transmission is stopped automatically when temperatures outside a treatment region reach a threshold. The ultrasound machine may provide a real-time visual feedback for monitoring the therapy, such as a 3D or multi-planar reconstruction (MPR) rendering of temperatures.

Various approaches to monitoring, adjusting HIFU beam focus location, and control by an ultrasound scanner of a therapy device or therapy may be used. These different approaches are used together or independently. In a first approach, a scanning sequence is automatically created on the ultrasound machine. The scanning sequence is used to control the timing for interleaving therapy dosing periods and monitoring periods. Acoustic radiation force imaging (ARFI) or similar ultrasound mode is used during the monitoring period. The scanning sequence dictates the starting time of the therapy, such as the therapy beam. Where the therapy device is a HIFU device, the scanning sequence may dictate the starting time of an ARFI push beam by the HIFU device. Triggering signals derived from the scanning sequence are generated by and sent from the ultrasound machine for and to a connected therapy device.

In a second approach, the scanning sequence and resulting triggering account for dithering of the treatment location. Multiple targets are stored in the HIFU device hardware. The ultrasound scanning sequence is also used to control the timing of stepping the HIFU beam to the different dithering (small movements) locations or targeting (arbitrary distances) locations.

In a third approach, a HIFU beam or other heat treatment focus is detected. Acoustic thermometry detects beam focus location. The focus is adjusted automatically with a closed loop temperature-based feedback. The closed loop may be driven by the scanning sequence, such as using the sequence to provide timing for the detection and/or adjustment.

In a fourth approach, visual HIFU beam focusing and therapy feedback is provided using real time live 3D and MPR rendering of the temperature. A graphics user interface for rendering manipulation is provided to the user.

In a fifth approach, a safety feature prevents unexpected heating outside the targeted region. The therapy, such as a HIFU beam, is shut down automatically if unexpected temperature or rise in temperature outside the targeting region is detected.

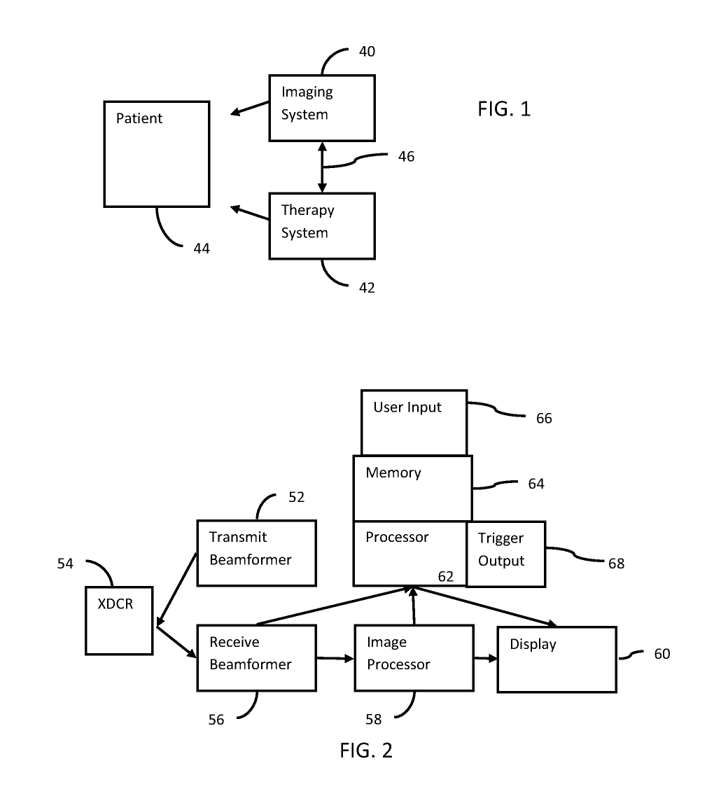

FIG. 1 shows one embodiment of a system using an ultrasound scanner for control and/or monitoring of therapy by a separate therapy device. The system operates in real time. Triggering, focal adjustment, rendering of temperature, and/or detection of heat outside a target region may operate in real-time with the therapy. The monitoring and/or control are provided in a closed loop cycling between the two operations during the treatment. The communications (e.g., triggering) from and the processing by the processor of the ultrasound scanner occur during treatment. The scanning for monitoring operates interleaved with the therapy, such as within fractions of a second or seconds of each other. In a same treatment session of a patient, the monitoring and/or control of the therapy device occurs.

The system includes an imaging system 40 and a therapy system 42 for use with the patient 44. The imaging system 40 is an ultrasound scanner, but other imaging systems may be used (e.g., magnetic resonance (MR)). The therapy system 42 is a HIFU system, microwave system, or other source of transmitted therapeutic energy. In one embodiment, the therapy system 42 includes a transducer, transmitters for generating waveforms to be applied to the transducer, electronics (e.g., processor) for controlling the therapy, and an interface for communicating, such as to receive trigger information and aiming information.

The imaging system 40 and therapy system 42 are separate systems. The coordinates of the systems 40, 42 are different. For example, separate transducers are used for therapy and imaging. The housings, electronics, or other components of the systems are separate.

The systems 40, 42 communicate to allow control. Location or coordinate information of the imaging system 40 is communicated to the therapy system 42. Timing or triggers are communicated from the imaging system 40 to the therapy system 42.

The communication is over the link 46. The link 46 is a cable. For example, a USB cable connects the therapy system 42 with the imaging system 40. Other types of cables may be used, such as an Ethernet cable. The link is direct (as shown without any intervening devices) or indirect, such as through a network or through a computer.

FIG. 2 shows one example ultrasound system 40 for control or monitoring of the therapy system 42. The ultrasound system of FIG. 2 includes a transmit beamformer 52, a transducer 54, a receive beamformer 56, an image processor 58, a display 60, a processor 62, a memory 64, a user input 66, and a trigger output 68. Additional, different or fewer components may be provided. For example, separate detectors and a scan converter are also provided.

The ultrasound system is a medical diagnostic ultrasound imaging system. Imaging includes two-dimensional, three-dimensional, B-mode, Doppler, color flow, spectral Doppler, M-mode or other imaging modalities now known or later developed. The ultrasound system is a full size cart mounted system, a smaller portable system, a hand-held system or other now known or later developed ultrasound imaging system. In one embodiment, the ultrasound system is a three-dimensional imaging system with the capability to receive along a plurality (e.g., 16, 32, 64 or more) receive lines in response to a given transmit beam for massively parallel receive beamforming to rapidly scan a volume.

The processor 62 and memory 64 are part of the ultrasound system, such as being a control processing unit and corresponding cache, RAM, system, or other memory. In another embodiment, the processor 62 and memory 64 are part of a separate system. For example, the processor 62 and the memory 64 are a workstation or personal computer operating independently of the ultrasound system. As another example, the processor 62 and the memory 64 are part of the therapy system 42.

The transducer 54 includes one or more imaging transducers. Any now known or later developed transducer for generating beams, fans, or other acoustic structure from electrical energy may be used. A single element may be provided, such as where focus is provided mechanically by movement or a lens. A plurality of elements in a one or multi-dimensional array may be used, such as an array of N×M elements where both N and M are greater than 1 for electric based focusing or steering.

The element or elements are piezoelectric, microelectromechanical, or other transducer for converting electrical energy to acoustic energy and acoustic energy to electrical energy. For example, the transducer 54 is a capacitive membrane ultrasound transducer.

The transducer 54 is operable from outside a patient. For example, the transducer 54 is a probe or other device held against the patient's skin. The transducer 54 is handheld, positioned by a device, or strapped to the patient. In other embodiments, the transducer 54 is in a probe, catheter or other device for operation from within a patient.

Each of the transducer elements connect to the transmit beamformer 52 for receiving electrical energy from the transmit beamformer 52. The transducer 54 converts the electrical energy into an acoustic beam for sampling. For reception, the elements connect with channels of the receive beamformer 56.

The imaging transducer 54 is separate from the therapy device, such as a HIFU transducer. The imaging transducer 54 may alternatively connect with the therapy transducer 34 in a fixed or flexible relationship. For example, the therapy and imaging transducers are in a cuff or blanket. The blanket is plastic, metal, fabric, or other material for rigidly, semi-rigidly or flexibly holding the plurality of transducers with or without the beamformers 52, 56, and/or processor 62. Hinges, other structure, or an outer casing interconnect the transducers.

The relative position of the therapy transducer or applicator and the imaging transducer 54 is measured, calibrated, registered, or fixed. Using the relationship, a transform between the coordinate space of the imaging system and the therapy device may be used to associate locations in both systems.

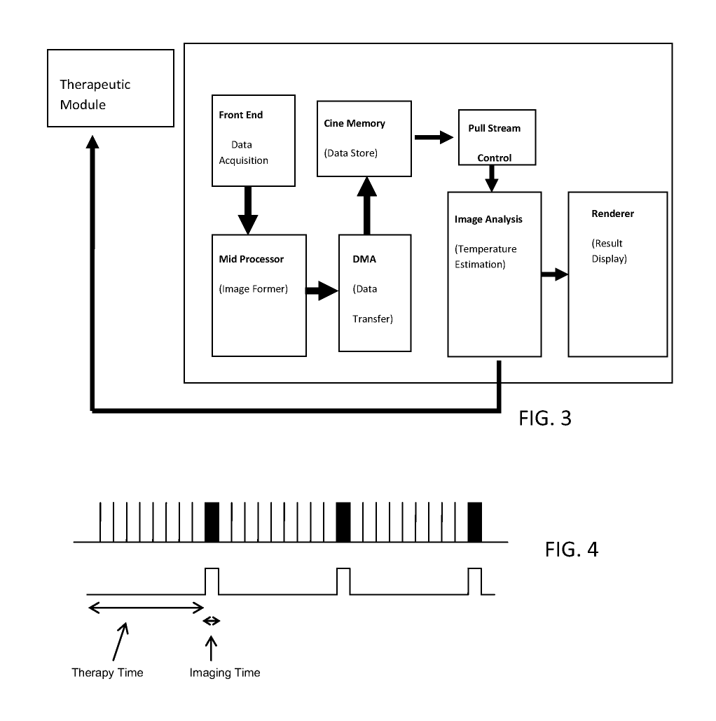

The beamformers 52, 56, image processor 58, and processor 62 are configured by hardware and/or software. Similarly, the therapy device may be configured by hardware and/or software. FIG. 3 represents software modules used in the system of FIG. 2. Other arrangements may be provided. The arrangement shown may be implemented on the SC2000 ultrasound machine by Siemens Medical Solutions.

FIG. 3 shows the interaction between different software modules on the ultrasound machine and the interaction between the ultrasound machine and the therapeutic module (i.e., therapy device). For imaging, software is provided for the front-end data acquisition. The software controls or configures the beamformers 52, 56. Software is provided for image forming and CINE memory operation. This software controls or configures the image processor 58 and/or memory 64. The module for the CINE operation may be separate. The software controls or configures the processor 62 for image analysis (e.g., temperature estimation) and rendering. Separate or the same devices and/or software modules may be used for image analysis and rendering. Software modules may be used for controlling the buffering, storage, or transfer of data, such as controlling data transfer automatically to the memory 64 or acquiring data from the memory 64 as needed for analysis or display. Other configurations may be used.

Referring again to FIG. 2, the transmit beamformer 52 is a transmitter of the ultrasound scanner. The transmit beamformer 52 connects with the imaging transducer 54. Electric waveforms are generated, such as with pulsers or waveform generators, for causing the transducer 54 to scan with a beam of ultrasound energy. The generators of the waveforms are transmitters. Repeating the transmissions in a pattern is a scan sequence. The front end software is responsible for programming the transmit beamformer 52 and receive beamformer 56, including a time controller, to generate the ultrasound image scan sequence and to acquire data for one or more ultrasound images.

The transmit beamformer 52 is one or more ultrasound memory, pulser, waveform generators, amplifiers, delays, phase rotators, multipliers, summers, digital-to-analog converters, filters, combinations thereof and other now known or later developed transmit beamformer components. The transmit beamformer 52 is configured into a plurality of channels for generating transmit signals for each element of a transmit aperture. The transmit signals for each element are delayed and apodized relative to each other for focusing acoustic energy along one or more scan lines. Signals of different amplitudes, frequencies, bandwidths, delays, spectral energy distributions or other characteristics are generated for one or more elements during a transmit event.

For imaging, the transmit beamformer 52 transmits a plurality of beams in a scan pattern. Upon transmission of acoustic waves from the transducer 34 in response to the generated waves, one or more beams are formed. A sequence of transmit beams are generated to scan a two or three-dimensional region. Sector, Vector®, linear, or other scan formats may be used. The same region is scanned multiple times. For flow or Doppler imaging and for strain imaging, an ensemble of scans is used for the same locations. In Doppler imaging, the sequence may include multiple beams along a same scan line before scanning an adjacent scan line. For strain imaging, scan or frame interleaving may be used (i.e., scan the entire region before scanning again). In alternative embodiments, the transmit beamformer 52 generates a plane wave or diverging wave for more rapid scanning.

The receive beamformer 56 is configured to acquire ultrasound data representing a region of a patient. The ultrasound data is for measuring temperature related information, acquiring anatomical information, detecting displacement, and/or receiving other data. The temperature and/or anatomical information are, at least in part, from ultrasound data.

The receive beamformer 56 includes a plurality of channels for separately processing signals received from different elements of the transducer 54. Each channel may include delays, phase rotators, amplifiers, filters, multipliers, summers, analog-to-digital converters, control processors, combinations thereof, and other now known or later developed receive beamformer components. The receive beamformer 56 also includes one or more summers for combining signals from different channels into a beamformed signal. A subsequent filter may also be provided. Other now known or later developed receive beamformers may be used. Electrical signals representing the acoustic echoes from a transmit event are passed to the channels of the receive beamformer 56. The receiver beamformer 56 outputs in-phase and quadrature (IQ), radio frequency or other data representing one or more locations in a scanned region. The channel data or receive beamformed data prior to detection may be used by the processor 62.

The receive beamformed signals are subsequently detected and used to generate an ultrasound image by the image processor 58. The image processor 58 is a B-mode/M-mode detector, Doppler/flow/tissue motion estimator, harmonic detector, contrast agent detector, spectral Doppler estimator, combinations thereof, or other now known or later developed device for generating an image from received signals. The image processor 58 may include a scan converter. The detected or estimated signals, prior to or after scan conversion, may be used by the processor 62.

The image processor 58 is a mid processor or image former. The various ultrasound beams represented by IQ data are converted to acoustic domain data for imaging. The processing between the output of the receive beamformer 56 to the input of the CINE memory is performed in the image processor 58. In one embodiment, various processing pipelines are used, such as for filtering, line interpolation, phase adjustment, amplification, or demodulation may be provided. For temperature estimation, one or more (e.g., all) of these processes may be bypassed. For example, the image processor 58 performs any coherent image processing (e.g., line interpolation or phase adjustment) and outputs IQ data to the memory 64 without detection or other operations of the image processor 58.

The IQ data may pass through no, one, or more buffers before output to the memory 64. For example, the data is buffered in an analytic data memory for storing IQ data before detection and in a detected data memory for storing detected data. The IQ data may be stored in the detected data memory despite not having been detected. Other buffering or no buffering arrangements may be used.

In one embodiment, the IQ data is associated with acoustic radiation force imaging (ARFI). Multiple firings are performed to acquire an ensemble of data represent each of a plurality of locations. To provide sufficient memory in the buffers or the memory 64, the memory 64 may be configured to reserve a sufficient bandwidth. For example, the CINE memory is configured to provide no or little set aside for Doppler or color data and/or B-mode data.

The memory 64 is a CINE memory in one embodiment. IQ, detected or other ultrasound data is stored in a loop structure. The most recent frames of data are stored. Once the amount, time or other limit is reached, the newest data replaces the oldest data. Frames of data are for complete one, two, or three-dimensional scans. A frame of data is a grouping of the data representing the scan region, such as the data from a sweep through a volume or ensemble data for multiple sweeps through a volume to generate a given image.

Alternatively or additionally, the memory 64 is a non-transitory computer readable storage medium having stored therein data representing instructions executable by the programmed processor for therapy monitoring or control with an ultrasound scanner. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The user input 66 is a button, knob, slider, touch pad, mouse, trackball, keyboard, or other now known or later developed input device. Combinations of input devices may be used. Based on a graphic user interface generated by the processor 62 and displayed on the display 60, the user uses the user input 66 to select, enter data, control, or configure the ultrasound scanner.

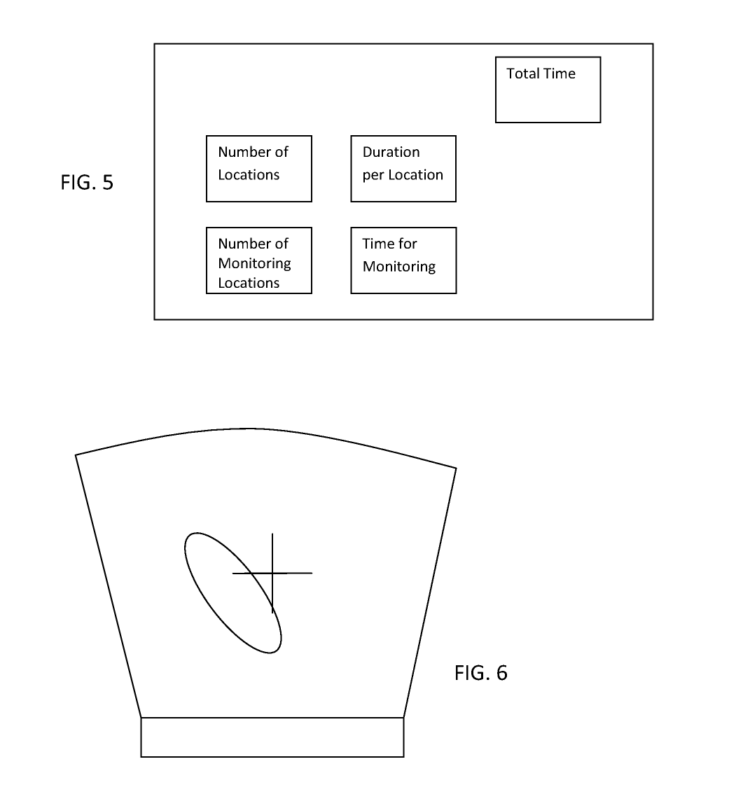

The user input 66 is part of a graphics user interface. In one embodiment, the user input 66 receives parameters of a therapy plan. FIG. 5 shows one example where the user enters a number of locations for a treatment region. The locations are for spatial offset of the treatment, such as for spatial dithering of the treatment. The duration at each location is input. The same or different durations are provided for different locations. The number of monitoring locations (e.g., scan pattern for sampling to image during therapy), the time for monitoring, and/or the total time to perform therapy and monitoring may be input. Additional, different, or fewer inputs for controlling the therapy and/or the monitoring may be provided. Transfer or loading of the therapy plan is provided in alternative embodiments.

The processor 62 is part of the ultrasound scanner, but may be a separate processor of a workstation, computer, or server connected with the ultrasound scanner. The processor 62 is a control processor, beamformer processor, general processor, application specific integrated circuit, field programmable gate array, digital components, analog components, hardware circuit, combinations thereof, or other now known or later developed devices for processing information. The processor 62 may be a single device for performing one or multiple operations or may be a group of devices for sequential or parallel processing.

In one embodiment, the processor 62 controls the transmit and receive beamformers 52, 56 or controls a controller for the transmit and receive beamformers 52, 56. The processor 62 creates the scan sequence for the ultrasound scanner. The scan sequence includes at least timing for transmitting. For example, given a depth of a field of view and a number (e.g., density and/or format) of transmit scan lines to use in sampling the field of view, a sequence of transmissions to scan the field of view is determined. The sequence includes a time between or for each transmit beam. More complicated scan sequences may be developed, such as associated with Doppler or flow imaging, M-mode imaging, spectral Doppler imaging (CW or PW), multiple pulse (e.g., harmonic imaging) or acoustic radiation force imaging or associated with combinations of different modalities. Transmit beams for different modes of imaging may be interleaved, resulting in variation in start times and intervals. In one embodiment, the sequence is created for a combination of B-mode and acoustic radiation force imaging. The B-mode portion of the sequence may be associated with a period over which a plurality of transmissions is regularly performed. The ARFI portion of the sequence may be a separate portion and associated with a pushing pulse of amplitude sufficient to move tissue and repetitive tracking scans along different lines to determine the timing and amount of displacement caused by the pushing pulse at different locations.

The scan sequence is a custom sequence. The custom sequence is stored, such as selecting from a plurality of options, or is generated by processing. Given a configuration of imaging modes and settings for the modes, the scan sequence for the particular configuration is created.

The sequence is customized to correspond to the therapy plan. The therapy plan defines the duration at each location, the number of locations, or other information. For example, a dose desired at each location and the number of locations may be defined. The therapy plan information is used to calculate the scan sequence. The therapy plan calls for transmissions being started at different times. The scan sequence is created to provide for transmissions or other scanning operations to occur at the desired start times for the therapy. Where the therapy is to dither locations, the scan sequence includes start times for the different locations and corresponding duration at each location.

The actual therapy firing sequence is done by the therapy device, not the ultrasound scanner. However, the ultrasound machine is responsible for controlling the timing of the therapeutic pulses. This is done by a pseudo imaging scan sequence on the ultrasound scanner. The pseudo imaging scan sequence includes multiple transmit firings with zero transmit power. While these transmit firings occurs, triggering signals are generated to synchronize the therapeutic firings.

The scan sequence is for configuring the ultrasound scanner, the transmit beamformer 52, and/or the receive beamformer 56. The scan sequence is for operation or scanning by the ultrasound scanner, but is created to correspond to the desired sequence for the therapy. For the parts of the scan sequence to be used for triggering therapy, the ultrasound scanner may be prevented from scanning despite being otherwise configured to do so by overriding the transmitters. The transmitters are turned off despite the configuration to scan. B-mode or other modes of imaging are used to emulate or include the therapy portions in the scan sequence.

For other portions of the scan sequence, the transmitters are allowed to operate. For the monitoring of the therapy, the ultrasound scanner is used. The transmitters operate to monitor the therapy, such as measure temperature or elasticity. For example, an ARFI sequence is used. The ARFI sequence is included in the custom sequence for actual operation of the ultrasound scanner. In one embodiment, the ultrasound scanner performs the entire sequence. In other embodiments, the pushing pulse or acoustic energy or beams used to displace tissue are generated by the therapy device. The transmitters are turned off for the pushing pulse despite the pushing pulse being in the sequence. For the acoustic energy (e.g., tracking pulses) used to monitor the displacement caused by the pushing pulse, the ultrasound scanner and corresponding transmitters are used.

The ARFI portion of the sequencing is designed so that the resultant ultrasound data may be processed to estimate the temperature in the scanning volume. The ARFI sequence is similar to that of the color mode. In each beam location, the ultrasound machine transmits and receives multiple times (e.g. 10 times). After a baseline scan (first of the firings in the ensemble), the push pulse is transmitted, such as by the therapy device based on a trigger from the scan sequence of the ultrasound scanner. After the push pulse, the remaining events for the ensemble occur (e.g., transmit and receive tracking pulses).

The processor 62 creates the scan sequence to schedule transmissions to occur based on the therapy plan and monitoring. The scan sequence includes transmission, reception or other events to occur in correspondence with the therapy plan and the monitoring despite the scan sequence being part of the ultrasound scanner for operation of the ultrasound scanner. The scan sequence interleaves the therapy and monitoring. Different portions of the sequence are used for monitoring and other portions are used for therapy. The monitoring portions may include portions for implementing by the therapy device and other portions for implementing by the ultrasound scanner, or all portions for monitoring are implemented by the ultrasound scanner.

FIG. 4 shows two trigger signals sent from the ultrasound scanner to the therapy system. The timing of both triggering signals is derived from the customized scan sequence. The lower trigger sequence is for configuring the type of transmission by the therapy device. The amplitude, duration, frequency, number of cycles in the waveform, or other characteristics of the transmission may be different for therapy verses ARFI pushing pulses. By indicating the timing for these different types, the therapy device may be configured to generate the desired type of acoustic energy. In alternative embodiments, only one type is generated by the therapy device or three or more types are provided. Given binary triggering, the triggers for different types may be indicated by order.

The upper sequence shows pulse triggers associated with the timing of the HIFU beam transmission. The regular spaced apart portions are for starting therapy. The dark blocks represent multiple triggers occurring close in time, such as associated with multiple pushing pulses for ARFI imaging. With greater temporal resolution, time between pulses in the block section is provided for monitoring transmissions by the ultrasound system. This sequence represents the scan sequence or selected portions of the scan sequence. The sequence is used as triggers for the therapy device. In the example of FIG. 4, the ARFI firings and therapy firings are interleaved. Any number of repetitions may be provided, such as until performance of the therapy plan is complete, a set time limit expires, or a temperature occurs.

The processor 62 controls the power to the transmitters or operation of the transmitters of the ultrasound scanner. For example, a switch for the control or driver signal to a pulser is opened to prevent operation. As another example, a source power is turned off or disconnected from the transmitter. The processor 62, directly or indirectly, turns off the transmitters for at least a portion of the scan sequence. In particular, the transmitters are turned off for the transmissions associated with the therapy plan and turned on for the transmissions associated with the monitoring. For example, the transmitters are turned off for the acoustic radiation force pulses and therapy pulses, but turned on for the tracking pulses. The tracking pulses from the ultrasound scanner scan tissue responsive to the acoustic radiation forces pulses. The tracking pulses occur within milliseconds of the pushing pulse in order to track tissue displacement.

The triggering signals are for controlling the external HIFU device. The ARFI tracking pulses are performed by the scan sequence inside the ultrasound scanner, so are not included or are filtered out of the trigger output.

The processor 62 may be configured to perform other functions, such as associated with a central processing unit or an image processor. For example, the processor 62 detects temperature from IQ or other ultrasound data in acoustic thermometry. Scan data acquired with the transmitters turned on is used to estimate temperature. Tissue expansion, speed of sound, or other characteristic of tissue is measured and used to determine temperature. Any now known or later developed thermometry may be used. For example, the processor 62 models an effect of thermal therapy on a treatment region. The temperature for one or more locations in the treatment region is estimated based on inputs to the model. The computer code implements a machine-learned model and/or a thermal model to estimate the temperature or temperature related information. The model is a matrix, algorithm, or combinations thereof to estimate based on one or more input features. In one embodiment, the processor 62 estimates temperature information as disclosed in U.S. Patent Application No. 2011/0060221, the disclosure of which is incorporated herein by reference. In other embodiments, the processor 62 uses measurements of one or more parameters to estimate temperature without a further code-based model.

The processor 62 may be configured to end the scan sequence and stop the triggers in response to a temperature measurement in a patient. The sequence may cease when a temperature of the treatment region reaches a certain point. A maximum temperature for treatment may be applied. The ceasing may be temporary, such as skipping one or more cycles of the therapy plan (e.g., stopping for milliseconds or one or more seconds), or be non-temporary, such as ceasing for the session.

The magnitude of the temperature is used. Alternatively, other characteristic are used. For example, the change or rate of change of temperature is used.

In one embodiment, the processor 62 monitors locations outside the treatment region. The treatment region includes all of the locations to be subjected to increased temperature for therapeutic effect. The treatment region may be just locations associated with application of the therapy (e.g., focal locations) or may include locations adjacent to focal locations. The lesion or other volume to be treated, with or without a margin, is the treatment region. A single location may be used as the treatment region.

The temperature is measured at the locations inside and/or outside the treatment region. Specific locations not in the treatment region may be identified. For example, organs to which limited temperature increase is desired are identified. For one or more locations outside the treatment region, the temperature is estimated as a safety feature. If the temperature exceeds (or meets) a threshold level, the scan sequence may be stopped. This temperature monitoring may avoid undesired heating outside the treatment region.

In another embodiment, the processor 62 is configured to control the focal location of the therapy device. For example, using temperature or tissue displacement measurement, the location of the focus is determined. The therapy device transmits the therapeutic energy (e.g., HIFU). The tissue is heated or moves as a result. The ultrasound scanner measures the effect of the application of therapy.

FIG. 6 shows an oblong region associated with increased or higher temperatures. This region corresponds to a focal location at the center with the therapy beam being along the long axis of the oblong region. The region of higher temperatures may have other shapes. The oblong region may be detected by thresholding, region shrinking, skeletonization, maximum temperature determination or other process. Alternatively, the location associated with the largest effect (e.g., largest increase in temperature or greatest temperature) is the focal location. Low pass filtering may be used for determining the location.

Based on user input, such as selection of a location in an image or images of an MPR, the treatment location or desired focus is known. Automatic detection, such as detection of a lesion by image processing, may instead be used. In FIG. 6, the desired focal location is represented by the cross-hairs. The desired focal location is offset from the actual focal location. As shown, the desired focal location is not within the region of increased temperatures, but may be. In other embodiments, the focal locations align (are at the same spot).

Any difference between the desired focal location or treatment region and the measured focal location is determined. Since the scan settings for depth, line spacing, and sample density are known from the scanning, the difference and direction between the focal location and the treatment location is determined using the ultrasound scanner. Based on calibration, known relationship, or calculated transform, the change in focus of the therapy device is determined and communicated to the therapy device. The communication is through coding of the triggering or a separate data communication.

The therapy device is controlled to adjust the focus to a desired location. Using repetition throughout treatment, the focus may be monitored and adjusted periodically. Alternatively, the focus is adjusted once or not at all. Likewise, a separate process of re-focusing the HIFU beam to ensure the HIFU beam focus location falls at the desired location may be performed before the treatment process. Control of the shape and/or size of the focus may also be performed.

The processor 62 may be configured for back end processing and rendering. Any image generation may be used. In one embodiment, the Open Inventor Programming model is used for MPR or three-dimensional rendering. The data flow and processing units are described in scene graphs. A scene graph is a collection of nodes or scene objects in a graph or tree structure. By operating the nodes or scenes while progressing through the tree, data representing a volume may be rendered for two-dimensional display. For example, a scene object which performs ARFI data analysis and temperature estimation is provided. Another scene object is provided for rendering. By following the scene graph, a rendering from three-dimensions of temperature information is created for display. Other rendering algorithms may be used.

The rendering is a surface, projection, or other rendering. In response to set or user input view direction or other characteristics, the rendering is performed. The user may select a subset of the volume to render, such as using a clipping plane. Given the viewing direction, a two-dimensional representation of the volume as viewed from the viewing direction is created.

In other embodiments, the rendering is an MPR. The user selects or set plane positions. The data, such as temperatures, representing the volume are interpolated or selected for the intersecting planes. Two or more planes are provided, such as three orthogonal planes centered and oriented as selected by the user. Multiple two-dimensional images representing the planes in the volume are rendered for viewing by the user.

The trigger output 68 is a port, such as a USB connector. The trigger output 68 is an output connector of the ultrasound scanner. The output may be dedicated to triggering or is a general output used for outputting triggering. In alternative embodiments, the trigger output 68 is a wireless transmitter for wireless communication of the trigger signals.

The trigger output 68 is connected with the therapy device for providing trigger signals, such as the signals shown in FIG. 4. The trigger signals are analog or digital. The trigger signals are derived from the scan sequence. By configuring the ultrasound scanner to scan a patient, triggers are generated by hardware and/or software from the scan sequence. These triggers are provided at the trigger output 68. Rather than reprogramming or generating of triggers separately for this application with a remote or separate therapy device, already existing trigger generation functions based on the scan sequence are used. In alternative embodiments, the trigger generation software or hardware is designed specifically for triggering therapy by a remote therapy device.

By customizing the scan sequence for the ultrasound scanner based on the therapy plan and including therapy transmissions by another device in the scan sequence, the triggers include start times and/or end times for operation of the therapy transmissions despite not being performed by the ultrasound scanner. The triggers interleave between therapy and monitoring triggers. The triggers may be distinguished by a separate triggering, such as the configuration trigger and the transmit trigger sequences of FIG. 4. The monitoring triggers are for generating a pushing pulse in ARFI. The therapy device may be used to generate the pushing pulses. In alternative embodiments, the triggers are just for therapy, such as where the ultrasound scanner generates the pushing pulses. Additional, different, or fewer triggers may be provided.

Triggers are not provided for the tracking pulses or other transmissions used to measure or transmissions of a lower power than the therapy or ARFI pushing pulse. The transmissions for imaging are performed by the ultrasound scanner.

The display 60 is a monitor, LCD, plasma, projector, printer, or other now known or later developed display device. The display 60 is configured to display an image representing the region of the patient and/or the effect of thermal therapy. For example, an anatomy image is displayed. As another example, temperature or related information is output as a value, graph, or two-dimensional representation. The processor 62 and/or the image processor 58 generate display signals for the display 60. The display signals, such as RGB values, may be used by the processor 62.

In one embodiment, the display 60 displays a rendering from data representing a volume. For example, the temperature data is surface or projection rendered from a view direction to a two-dimensional representation (e.g., to a view plane). As another example, the temperature data is interpolated or selected for multiple planes in the volume for rendering an MPR. Multiple two-dimensional images representing the different planes are displayed. The renderings represent the spatial distribution of temperature. This temperature information may be used for adjusting focus, monitoring therapy, verifying desired temperatures at different locations (e.g., lower temperatures outside the treatment region and higher temperatures in the treatment region) or other monitoring by the user. The progression of therapy, such as increases in temperature at different locations due to dithering, may be viewed through a succession of images.

The images are in real time. As scan data is acquired and temperatures estimated, the images are generated. The images have a one second or less delay from completion of scanning for the data used in the image to display of the image. The images represent the effect of therapy and/or patient as (e.g., within a seconds) the therapy occurs. In alternative embodiments, the images are generated after scanning or the imaging session has ended.

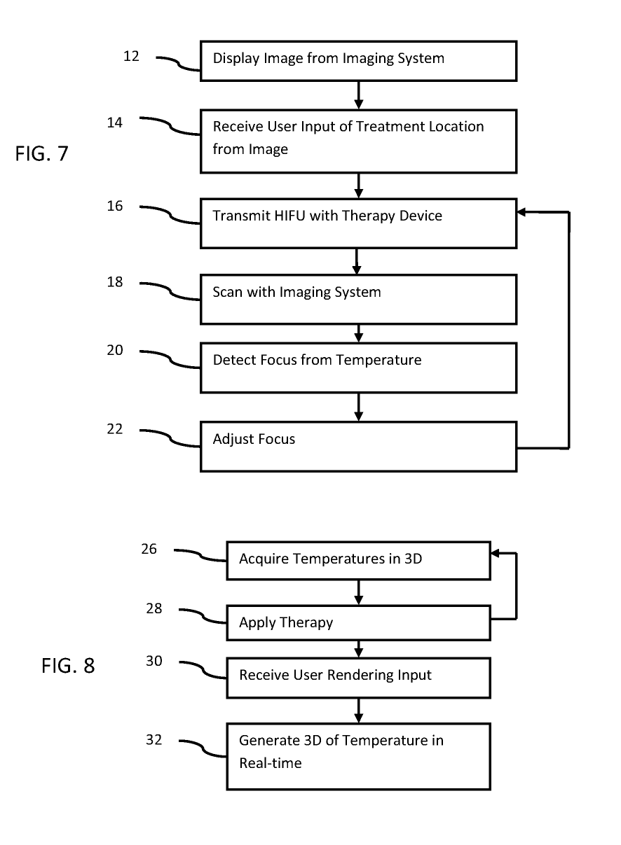

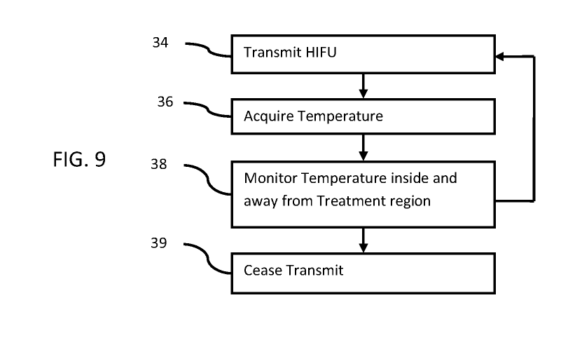

FIG. 7 shows one embodiment of a method of therapy monitoring with an ultrasound scanner. The method provides for maintaining the focus of therapy at the treatment locations. The method is implemented using the system of FIG. 1 or 2, or a different system. Additional, different, or fewer acts may be provided. For example, acts 12 and/or 14 are optional. As another example, acts from FIGS. 8 and/or 9 may be included. In one embodiment, the acts of FIG. 7 are performed in a loop to periodically adjust the focal location of the therapy. The acts of FIG. 8 are performed during or between periodic performances of the acts of FIG. 7, such as providing images to the user based on the acts of FIG. 8 and using those images or automated detection to adjust the focus using the acts of FIG. 7. The safety monitoring acts of FIG. 9 are performed periodically, such as the same or different period as the acts of FIG. 7. The acts are performed in the order shown or a different order.

The acts are performed for therapy. In a therapy session for a given patient, the patient is readied for the therapy. A sonographer or physician places therapy and imaging transducers on the patient. Before the high intensity focused ultrasound (HIFU) or other therapy begins for ablation or other treatment, the spatial relationship of the transducers is determined. The spatial relationship may be used to transform coordinates between the ultrasound imaging system and the therapy system.

In act 12, an image is displayed. The image represents a one, two, or three-dimensional region. The image is a strain, elastography, B-mode or other image. In one embodiment, the image is an MPR from B-mode data.

In act 14, the user identifies a treatment location. The lesion or other location for treatment is selected by the user. The selection may be a point, a line, an area, or a volume. For example, a point or area may be selected using a user interface on each of three orthogonal planes of an MPR. The selections are converted into a volume treatment region. In alternative embodiments, the processor detects a treatment region using data processing.

Once the treatment region is identified, treatment may begin. The location of the treatment region is communicated to the therapy device. The coordinates in the therapy system for the treatment location of the patient are determined from the transformation from the coordinates of the region selected based on the images of the ultrasound imaging system. The therapy plan is input and communicated to the therapy device, or input to the therapy device and communicated to the imaging system. The dose, angle, focus, and/or other characteristics of the therapy are established for the treatment location. The location of the focus, origin, scan line, or application of the therapy is based on treatment region and therapy plan. The therapy device is configured for treatment based on triggers from the ultrasound system. Once configured, the treatment may begin.

In act 16, a therapy waveform is transmitted. In the HIFU embodiment, high intensity focused ultrasound therapy waveforms are transmitted. High voltage waveforms are applied to the therapy ultrasound transducer, which generates the HIFU therapy waveforms in the acoustic domain. The HIFU pulse or pulses are focused using a phased array and/or mechanical focus and provide the high intensity acoustic energy to tissue at a focal or beam location. The acoustic energy is focused, resulting in a three-dimensional beam profile with a focal location at a depth along the beam. The focus may be fixed or steerable. The excitation may be unfocused in one dimension, such as the elevation dimension. The excitation is transmitted into tissue of a patient. For a given transmission, a single beam is formed. Alternatively, multiple beams with respective foci are formed for a given transmission.

The therapeutic ultrasound pulse has a plurality of cycles at any desired frequency and amplitude. In one embodiment, the therapeutic pulse lasts for a fraction of a second to seconds at an ultrasound frequency, such as 500 KHz-20 MHz. Any peak intensity may be provided, such as 100 or more watts per square centimeter, 500 or more watts per square centimeter, 1000-2000 watts per square centimeter, or about 1000 watts per square centimeter. Any now known or later developed therapeutic waveform with any intensity, frequency, and/or number of cycles may be used. The waveform is continuous or intermittent.

The therapeutic ultrasound pulse treats the tissue by generating heat at the desired tissue location. The intensity also generates stress on the tissue. The pulse pushes the tissue towards and away from the transducer with negative and positive acoustic pressures. For a sufficiently long therapeutic pulse, a substantially constant strain on the tissue is created. The strain, c, is a function of the tissue stiffness, E, the viscosity, η, and the stress from HIFU radiation force. The steady state stress during the therapeutic pulse is proportional to the ratio of average HIFU intensity, I, to the speed of sound in the tissue, c.

The HIFU waveforms may also generate biomechanical changes. The thermal effects of the therapy acoustic energy may cause changes in volume due to thermal expansion, in the speed of sound (c), in tissue stiffness (E), and/or in the viscosity (η) of fluids in the tissue. The therapy acoustic energy may also induce mechanical effects, such as radiation pressure, streaming, and/or cavitations. The biological effects may include hyperthermia at tissue temperature of about 41-45° C., protein denaturation at temperatures above 45° C., and tissue necrosis at temperatures above 50° C. Tissue stiffness may be affected even at temperatures below 45° C. At temperatures above 45° C., increases in viscosity and/or stiffness may occur. At temperatures above 50° C., the tissue may have a high stiffness and/or high attenuation.

In act 18, the imaging system scans the patient to detect the tissue response to the therapy sample. Any type of scan, scan format, or imaging mode may be used. For example, harmonic imaging is used with or without added contrast agents. As another example, B-mode, color flow mode, spectral Doppler mode, M-mode, or other imaging mode is used. Any mode of magnetic resonance may be used.

Data representing anatomical or other information is acquired from the patient. The data represents a point, a line, an area, or a volume of the patient. For ultrasound imaging, waveforms at ultrasound frequencies are transmitted, and echoes are received. The acoustic echoes are converted into electrical signals and beamformed to represent sampled locations within a region of the patient. The beamformed data may be filtered or otherwise processed. The beamformed data may be detected, such as determining intensity. A sequence of echo signals from a same location may be used to estimate velocity, variance, and/or energy. Echoes at one or more harmonics of the transmitted waveforms may be processed. The detected values may be filtered and/or scan converted to a display format. The ultrasound data representing the patient is from any point along the ultrasound processing path, such as channel data prior to beamformation, radio frequency or in-phase and quadrature data prior to detection, detected data, or scan converted data.

Data may be derived from acquired data. For example, the types of tissue at different locations is determined from a speckle characteristic, echo intensity, template matching with tissue structure, or other processing. As another example, region growing is used with B-mode data or color flow data to determine that the ultrasound data represents a vessel or other fluid region. A current distribution of anatomy, such as a list of represented organs, may be determined.

The scan occurs during application of the therapy or after application. For example, the scanning to detect temperature or temperature change may occur after transmission of the therapy beam but before temperature equalization. As another example, the scanning occurs prior to and immediately after transmission of the therapy beam to detect displacement of the tissue or change of temperature.

The scanning is for thermometry. By performing thermometry by scanning and detecting, the temperature or change of temperature of various locations may be determined. Thermometry images or data are used to detect the temperature or temperature rise within the field of view associated with the sample.

Any temperature related measurement may be used. Ultrasound measurements may be provided for a plurality of different locations. Any now known or later developed temperature related measurement using ultrasound may be used. For example, tissue expands when heated. Measuring the expansion may indicate temperature. Temperature related measurements may directly or indirectly indicate a temperature. For example, a measure of a parameter related to conductivity or water content (e.g., a measurement of the type of tissue) may indirectly impact the temperature. The measurements may be for raw ultrasound data or may be derived from ultrasound data. In one embodiment, two or more, such as all four, of tissue displacement, speed of sound, backscatter intensity, and a normalized correlation coefficient of received signals are performed. Other measurements are possible, such as expansion of vessel walls.

Tissue displacement is measured by determining an offset in one, two, or three-dimensions. A displacement associated with a minimum sum of absolute differences or highest correlation is determined. The current scan data is translated, rotated, and/or scaled relative to a reference dataset, such as a previous or initial scan. The offset associated with a greatest or sufficient similarity is determined as the displacement. B-mode or harmonic mode data is used, but other data may be used. The displacement calculated for one location may be used to refine the search or search region in another location. Other measures of displacement may be used.

The speed of sound may be measured by comparison in receive time from prior to heating with receive time during heating. A pulse is transmitted. The time for the echo to return from a given location may be used to determine the speed of sound from the transducer to the location and back. Any aperture may be used, such as separately measuring for the same locations with different apertures and averaging. In another embodiment, signals are correlated. For example, in-phase and quadrature signals after beamformation are correlated with reference signals. A phase offset between the reference and current signals is determined. The frequency of the transmitted waveform (i.e., ultrasound frequency) is used to convert the phase difference to a time or speed of sound. Other measurements of the speed of sound may be used.

The backscatter intensity is B-mode or M-mode. The intensity or energy of the envelope of the echo signal is determined.

The normalized correlation coefficient of received signals may be measured. Beamformed data prior to detection, such as in-phase and quadrature data, is cross-correlated. In one embodiment, a reference sample or samples are acquired. During or after transmission of the sample, subsequent samples are acquired. For each location, a spatial window, such as three wavelengths in depth, defines the data for correlation. The window defines a length, area or volume. The current data is correlated with the reference data within the window space. The normalized cross-correlation is performed for the data in the window. As new data is acquired, further cross-correlation is performed.

Any temperature associated acoustic and physical parameters or changes in the parameters may be measured. Other measurements include tissue elasticity, strain, strain rate, motion (e.g., displacement or color flow measurement), or reflected power (e.g., backscatter cross-section).

In one embodiment, the temperature is estimated from a model rather than directly measured. One or more of the types of information discussed above may be used as inputs to the model. The actual data and/or derived information are anatomical parameters to be used in combination with the model. In addition to the ultrasound scanning, clinical or other information may be acquired for determining the temperature. For example, genetic information or other tissue related data may be mined from a patient record. Any feature contributing to determination of temperature related information may be used.

Expansion, shrinkage, water content, or other therapy parameters may indicate a current temperature. Regardless of the categorization of the measurement, the measurements are used as inputs to a model or to calculate values for input to the model. The data is provided for one or more locations, such as providing data for all locations in a two- or three-dimensional region. Alternatively, the data is generally associated with the entire region, such as one dose or energy level for the entire region.

The temperature related measurements are applied to a model. The measurements or data are input as raw data. Alternatively, the values (i.e., measurements and/or data) are processed and the processed values are input. For example, the values are filtered spatially and/or temporally. As another example, a different type of value may be calculated from the values, such as determining a variance, a derivative, normalized, or other function from the values. In another example, the change between the current values and reference or previous values is determined. A time-history of the values over a window of time may be used. The values are input as features of the model.

The output of the model may be used as an input. For an initial application of the model, the feedback is replaced with a reference temperature, such as the temperature of the patient. For further application of the model, the previous output is fed back as an input, providing a time-dependent model. The temperature related information output by the model is fed back as a time history of the information, such as temperature at one or more other times. The measured or received values are updated (i.e., current values are input for each application of the model), but previous values may also be used. The feedback provides an estimated spatial distribution of temperature or related information in the region at a previous time. The subsequent output of the model is a function of the ultrasound data or other values and a previous output of the modeling. The time-history of the values may be used as inputs, such that the time history and spatial distributions of the temperature-associated and therapeutic effect-related parameters are used as features of the model. In alternative embodiment, no feedback is used.

The model outputs a temperature or temperature distribution (i.e., temperature at different locations and/or times) from the input information. The derived temperature may be in any unit, such as degrees Fahrenheit or Celsius. The resolution of the temperature may be at any level, such as outputting temperature as in one of multiple two or other degree ranges. Alternatively, other temperature related information is output, such as a change in temperature, a dose, or an index value.

Any model may be used, such as a neural network or a piecewise linear model. The model is programmed or designed based on theory or experimentation. In one embodiment, the model is a machine-learned model. The model is trained from a set of training data labeled with a ground truth, such as training data associated with actual temperatures. For example, the various measures or receive data are acquired over time for each of multiple patients. During transmission of the sample therapy, the temperature is measured. The temperature is the ground truth. Through one or more various machine-learning processes, the model is trained to predict temperature given the values and/or any feedback.

Any machine-learning algorithm or approach to classification may be used. For example, a support vector machine (e.g., 2-norm SVM), linear regression, boosting network, probabilistic boosting tree, linear discriminant analysis, relevance vector machine, neural network, combinations thereof, or other now known or later developed machine learning is provided. The machine learning provides a matrix or other output. The matrix is derived from analysis of a database of training data with known results. The machine-learning algorithm determines the relationship of different inputs to the result. The learning may select only a sub-set of input features or may use all available input features. A programmer may influence or control which input features to use or other performance of the training. For example, the programmer may limit the available features to measurements available in real-time. The matrix associates input features with outcomes, providing a model for classifying. Machine training provides relationships using one or more input variables with outcome, allowing for verification or creation of interrelationships not easily performed manually.

The model represents a probability of temperature related information. This probability is likelihood for the temperature related information. A range of probabilities associated with different temperatures is output. Alternatively, the temperature with the highest probability is output. In other embodiments, the temperature related information is output without probability information.

As an alternative to machine learning, manually programmed models may be used. The model may be validated using machine training. In one embodiment, a thermal distribution model is used. The thermal distribution model accounts for the thermal conductivity, density, or other behavior of different tissues, fluids, or structures. The thermal distribution model receives temperatures, temperature related information, measurements, or other data. The input information may be sparse, such as having temperature information for one or more, but fewer than all locations. The thermal distribution model determines the temperature at other locations. The thermal distribution model may determine the temperature at other times or both time and location.

In another embodiment, the thermal distribution model corrects temperatures based on anatomy. For example, a machine-learned model estimates temperature for uniform tissue. The temperature output is corrected to account for tissue differences in the region, such as reducing the temperature around thermally conductive vessels or fluid regions.

In response to input of the features, the model outputs the temperature related information, such as temperature. The spatial distribution of temperature is used to identify a focal region within the imaging device's coordinates in act 20. Measurements are performed for multiple locations in a region. Full or sparse sampling may be used. The measurements are performed over time, but independent of previous measurements. Alternatively or additionally, a change in a measurement from a reference or any previous (e.g., most recent) measurement may be used.

Non-real time measurements may be used, such as a baseline temperature. MRI-based measurements for temperature distribution in a region may be used. Real-time measurements may be used, such as associated with ultrasound measurements performed during application of thermal therapy to a region of the patient.

In act 20, a beam location or focal region for the HIFU is determined using the temperature or other characteristic. The therapy focus is detected using acoustic thermometry. Locations associated with sufficient magnitude, change of temperature, or rate of change are identified. Locations where the displacement, temperature, or temperature change is relatively high are identified by applying a threshold. The threshold may be preprogrammed or adapted to a given data set. The threshold may be normalized, such as a threshold based on data at spatial locations spaced away from the likely location of the beam or focal region. As another example, an average or other percentage displacement or temperature across a region of interest is determined. The temperature data may or may not be spatially filtered prior to application of the threshold.

Locations associated with a temperature greater than the average or other percentage indicates beam or focal location generally. The temperatures or locations of sufficient temperature may be low pass filtered after application of the threshold and before identifying the focal region from the remaining temperature data.

A center of a largest region of increased temperature, region growing, region shrinking, or other processes may be used to find the focal point. The point may be determined with or without segmentation. The point may be a center of gravity for a region, such as the largest segmented region of increased temperature. In one embodiment, the point is a maximum temperature value (e.g., highest echo strain value) in a region of higher temperature values. The focal location is a location of greatest temperature or change in temperature relative to surrounding locations in a volume. The temperatures may be low pass filtered in addition to any other filtering before determining the location of the maximum or center.

In act 22, the focus of the therapy is adjusted. This adjustment is in addition to any adjustment as part of the sequencing of the therapy plan. For example, the desired focal location may shift over time. The adjustment of act 22 is to align the actual focus with the current desired focus.

The difference between the detected and desired focal points is determined. The difference is a distance and direction, such as a two or three-dimensional vector. The difference is a spatial difference.

The ultrasound imaging system determines whether the therapy beam focus is close enough to the targeting position or desired focus. The threshold for close enough may be predetermined or adaptive. For example, close enough may be within 5 mm. As another example, close enough near some organs may be different than close enough near other organs. The size of the treatment region may be used, such as within a % of the diameter of the treatment region being close enough.

The ultrasound imaging system provides guidance to the therapy system on steering the beam closer to the targeting location. The difference indicates an adjustment. Based on the transform between the imaging and therapy systems, the difference detected by the imaging system between the desired and temperature-detected focal points is converted into a difference for the therapy system. The focus of the therapy system is adjusted based on this feedback.

Acts 16, 18, and 20 may be repeated. The HIFU may be continuous or sporadic. Any treatment regimen may be used. During ongoing treatment or in between different fractions of the treatment, the transmission of act 16, imaging of act 18, and detection of act 20 may be repeated. Where the focus is to be adjusted, act 22 is repeated.

FIG. 8 shows one embodiment of a method for monitoring treatment. The treatment is monitored by the user. The user may adjust the imaging in real-time to view the effects of therapy. Once the therapy beam focus tuning process is finished (see FIG. 7), the ultrasound imaging system starts interleaved therapy and monitoring sequencing. Additional, different, or fewer acts may be provided. The acts are performed in the order shown or a different order.

In act 26, temperatures of tissue in locations distributed in three-dimensions are acquired. The temperature is measured with ultrasound, such as using the model. Values representing temperature at different locations are obtained. The locations represent a volume, such as a volume of the patient around and including the treatment region. The volume may be only the treatment region.

In act 28, therapeutic transmissions are performed. Therapy is applied. The therapy is interleaved with the measurement of temperatures. Any interleaving may be used, such as treating one location, then monitoring temperature, then treating another location and so on. The interleaving may divide the treatment for a given location into different portions with the monitoring scans occurring in-between portions.

In act 30, user input is received. Signals from a user interface, such as a touch screen, mouse or trackball and button, or arrow keys, are received.

The user indicates a viewing direction (angle), a plane position, a segmentation, a cutting plane, or other rendering information. The input is to control rendering. To view from different perspectives, control the information being viewed, or to examine the effects of treatment, the rendering is controlled by the user.

In act 32, a rendering is generated. The rendering is a two-dimensional representation of the volume of the patient. Surface or projection rendering may be used. For this three-dimensional rendering, the volume may be viewed from any angle. The user selected angle is used, allowing the user to view from different or a desired direction. This may allow the view to focus on particular organs or locations in the patient.

Similarly, the rendering may be an MPR. By selecting one or more cut-plane positions, the user controls the information displayed on the two-dimensional display.

The rendering is of temperatures. The temperatures within the volume are used to modulate the color, brightness, or other characteristic of the image. For example, mapping modulates the color as a function of the temperature related information, such as the shade of red or color between red and yellow being different for different temperatures. The change or rate of change in temperature may alternatively be mapped to the output color or additionally mapped to brightness or other aspect of the color.

The rendering is generated in real-time with the therapeutic transmissions. As data is acquired, images are rendered. The effects of therapy may be immediately viewed.

Other information may be displayed as well. For example, the temperatures are rendered as color overlaid on a rendering from B-mode data. The temperatures for different anatomy or locations in tissue are represented. The overlay is laid over an ultrasound image representing the anatomy, such as overlaid on a B-mode image. The overlay is spatially and temporally registered to the anatomic information. The overlay indicates temperatures caused by the therapy system and the underlying anatomical image may show the anatomy to be treated.

The image may include other temperature related information. The temperature related information is displayed as a value, such as a temperature or dose. A graph of temperature as a function of time or along a line may be displayed.

FIG. 9 shows one embodiment of a method for controlling treatment. The treatment is controlled by the ultrasound imaging system. The treatment is monitored to avoid heating of tissue to undesired levels. In particular, heating to therapeutic levels of tissue not to be treated is avoided. While the therapy and monitoring by the user (see FIG. 8) are on-going, the imaging system may automatically monitor the temperatures. Additional, different, or fewer acts may be provided. The acts are performed in the order shown or a different order.

In act 34, the therapy beams are transmitted. For example, HIFU treatment is applied. The treatment is of a treatment region. The focus of the treatment is at one location or a plurality of locations in the treatment region.

In act 36, the temperatures for a volume are acquired. The volume includes the treatment region and locations outside the treatment region. Any size of the region beyond the treatment region may be included. For example, the treatment region is centered in the scan volume and encompasses about 10-50% of the volume. Other ratios of treatment to non-treatment regions may be used. Alternatively, temperature is estimated only for locations outside the treatment region.

In act 38, the temperatures are monitored. The temperatures in the treatment region may be monitored. For example, the dosage is measured. As another example, a maximum temperature may be avoided by monitoring the treatment region.

The temperatures outside the treatment region are monitored. All the locations outside the treatment region are monitored. Alternatively, just locations within a range of the treatment region are monitored. In one embodiment, locations associated with tissue at risk are monitored. For example, the user inputs selection of one or more restricted locations and corresponding temperature limits. Any criteria may be used for selecting one or more locations to monitor.

The monitoring is performed by the ultrasound imaging system. The monitoring is automated. A threshold temperature, change in temperature, rate of change in temperature, or combinations thereof is compared to the temperature information for the locations. The threshold is set to avoid any or a particular biological effect.

In act 39, the therapy transmissions are ceased in response to the monitoring. If the temperature at a location outside the treatment region reaches or exceeds the threshold, the therapy may be stopped. The ceasing may be for a fraction of the therapy plan. The ceasing may be for a location, such as shifting the treatment to a different location in the treatment region. The ceasing may be temporary, such as allowing user adjustment of the therapy plan or allowing cooling of the tissue. The ceasing may be for the reminder of the therapy session.

The temperature information for one location may be used to cease the therapy. Any location exceeding the threshold for that location triggers stoppage. Alternatively, the temperature information for a plurality of locations exceeding the corresponding thresholds occurs before ceasing.

The ultrasound imaging system obtains temperature information in the volume of interest. If the temperature has unexpected temperature rise outside the treatment region, the interleaved therapy and monitoring sequencing is stopped. Otherwise, the interleaved therapy and monitoring sequencing continues or is repeated.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for therapy control with an ultrasound scanner, the system comprising:
transmitters of the ultrasound scanner, the transmitters operable to scan with beams of ultrasound in a scan sequence;
a processor of the ultrasound scanner, the processor configured to create the scan sequence for the ultrasound scanner as a function of a therapy plan and to turn off the transmitters for at least a portion of the scan sequence; and
a trigger output of the ultrasound scanner configured to output triggers to a therapy device, the output triggers responsive to the scan sequence.

2. The system of claim 1 further comprising:
a user input operable to receive parameters of the therapy plan, the parameters including a number of locations, a duration, or both the number of locations and the duration.

3. The system of claim 1 wherein the processor is configured to create the scan sequence to schedule transmissions to occur based on a duration in the therapy plan.

4. The system of claim 1 wherein the processor is configured to create the scan sequence to schedule transmissions to occur based on the therapy plan and monitoring, the transmitters turned off for the transmissions associated with the therapy plan and turned on for the transmissions associated with the monitoring.

5. The system of claim 1 wherein the scan sequence includes acoustic radiation force pulses and tracking pulses, the transmitters turned off for the acoustic radiation force pulses and turned on for the tracking pulses;
wherein the triggers are for generation of the acoustic radiation force pulses by the therapy device with the tracking pulses from the ultrasound scanner scanning tissue responsive to the acoustic radiation forces pulses.

6. The system of claim 1 wherein the scan sequence configures the ultrasound scanner to scan but for the transmitters being off.

7. The system of claim 1 wherein the triggers interleave between therapy and monitoring triggers.

8. The system of claim 1 wherein the scan sequence indicates a start time of a therapy beam, an acoustic radiation force push beam, and monitoring beams, and wherein the triggers are for the therapy beam and the acoustic radiation force push beam and not the monitoring beams.

9. The system of claim 1 wherein the scan sequence includes different periods for location dithering of therapy beams by the therapy device.

10. The system of claim 1 wherein the processor is configured to end the scan sequence and stop the triggers in response to a temperature measurement in a patient and outside of a treatment region.

11. The system of claim 10 wherein the temperature measurement is responsive to scanning by the ultrasound scanner in the scan sequence with the transmitters on.

12. The system of claim 1 wherein the processor is configured to determine a focus of the therapy device from temperature measured with ultrasound and transmit a location to the therapy device.

13. The system of claim 1 further comprising:
a display operable to display a three-dimensional or multi planar reconstruction image of temperature in a patient.

14. The system of claim 1 wherein the processor and trigger output are operable in real-time with treatment by the therapy device.

15. A method for therapy control with an ultrasound scanner, the method comprising:
identifying a treatment location;
detecting a focus of a heat therapy with acoustic thermometry; and
adjusting the focus to be at the treatment location based on the detection with the acoustic thermometry.

16. The method of claim 15 wherein detecting comprises transmitting high intensity focused ultrasound and detecting the focus as a first location of greater temperature than surrounding locations in a three-dimensional volume.

17. In a non-transitory computer readable storage medium having stored therein data representing instructions executable by a programmed processor for therapy monitoring with an ultrasound scanner, the storage medium comprising instructions for:
acquiring temperatures of tissue in locations distributed in three-dimensions;
interleaving therapeutic transmissions with the acquiring of the temperatures;
receiving user input of rendering manipulation; and
generating, in real time with the therapeutic transmissions, a three-dimensional or a multi-planar reconstruction rendering to a two-dimensional image of the temperatures, the rendering being a function of the rendering manipulation.

18. The non-transitory computer readable storage medium of claim 17 wherein receiving comprises receiving a viewing angle and wherein generating comprises generating the three-dimensional rendering as a function of the viewing angle.

19. The non-transitory computer readable storage medium of claim 17 wherein receiving comprises receiving a cut plane selection and wherein generating comprises generating the multi-planar reconstruction rendering as a function of the cut plane selection.

20. In a non-transitory computer readable storage medium having stored therein data representing instructions executable by a programmed processor for therapy control with an ultrasound scanner, the storage medium comprising instructions for:
transmitting therapeutic high intensity focused ultrasound to a treatment region within a patient region;
acquiring temperatures of tissue in locations distributed in the patient region, at least some of the locations outside of the treatment region; and
ceasing the transmitting of the therapeutic high intensity focused ultrasound based on at least one temperature for at least one of the locations outside the treatment region.

21. The non-transitory computer readable storage medium of claim 20 wherein ceasing comprises ceasing where the at least one temperature is above a threshold, the threshold being below a biological effect threshold of tissue response to heat.
